(19) United States
(12) Patent Application Publication
(10) Pub. No.: US 2018/0299448 A1
(43) Pub. Date: Oct. 18, 2018

(54) COLORECTAL CANCER SCREENING METHOD

(71) Applicants: SHIMADZU CORPORATION, Kyoto-shi (JP); NATIONAL UNIVERSITY CORPORATION KOBE UNIVERSITY, Kobe-shi (JP); NATIONAL CANCER CENTER, Tokyo (JP)

(72) Inventors: Noriyuki OJIMA, Kyoto-shi (JP); Shuichi KAWANA, Kyoto-shi (JP); Yumi UNNO, Kyoto-shi (JP); Takero SAKAI, Kyoto-shi (JP); Kenichi OBAYASHI, Kyoto-shi (JP); Yukihiko KUDO, Kyoto-shi (JP); Masaru YOSHIDA, Kobe-shi (JP); Shin NISHIUMI, Kobe-shi (JP); Takashi KOBAYASHI, Kobe-shi (JP); Takeshi AZUMA, Kobe-shi (JP); Koji OKAMOTO, Tokyo (JP); Yasuhide YAMADA, Tokyo (JP); Natsuko OKITA, Tokyo (JP); Kazuki SUDO, Tokyo (JP)

(73) Assignees: SHIMADZU CORPORATION, Kyoto (JP); NATIONAL UNIVERSITY CORPORATION KOBE UNIVERSITY, Kobe-shi (JP); NATIONAL CANCER CENTER, Tokyo (JP)

(21) Appl. No.: 15/887,011

(22) Filed: Feb. 2, 2018

(30) Foreign Application Priority Data
Mar. 30, 2017 (JP) 2017-069354

(51) Int. Cl. G01N 33/574; G01N 33/49

(52) U.S. Cl. CPC G01N 33/57419; G01N 33/492

(57) ABSTRACT

Provided is a colorectal cancer inspection method by which the presence of early colorectal cancer at any one of stages from 0 to 2 can be accurately determined. The colorectal cancer inspection method includes: measuring the amounts of at least lactic acid, pyruvic acid, and glycolic acid among a plurality of kinds of in vivo metabolites contained in a biological sample collected from a test subject, based on data obtained by performing a chromatograph-MS/MS analysis on the biological sample; and determining the presence of colorectal cancer at any one of stages from 0 to 2, based on a measurement value of at least one of the lactic acid, the pyruvic acid, and the glycolic acid.

### TECHNICAL FIELD

The present invention relates to a colorectal cancer inspection method using an analysis result obtained by quantitatively analyzing in vivo metabolites in a biological sample collected from a test subject.

### BACKGROUND ART

It is known that early colorectal cancer can be cured with a high degree of probability. However, colorectal cancer has no subjective symptoms at its early stages. When subjective symptoms do appear, the condition of the colorectal cancer is often at its more serious stages. To address this problem, a screening test for discovering if a patient is suffering from early colorectal cancer is conducted in typical regular health examinations for healthy individuals in hospitals or similar medical institutions.

The screening is typically conducted through a colorectal cancer inspection which is called the fecal occult blood test. In the fecal occult blood test, an extremely small quantity of blood (occult blood) in feces, which is caused by bleeding from an alimentary canal, is detected by an immunologic procedure using an anti-human hemoglobin antibody. A subject who has tested positive by the fecal occult blood test (i.e., who is suspected of suffering from colorectal cancer) subsequently undergoes a digital rectal examination, endoscopy, contrast-X-ray examination, or similar complete examination. The results of these examinations are comprehensively judged by medical experts, such as doctors, to determine the presence of colorectal cancer.

High levels of determination sensitivities are naturally required for the screening, whereas it is mainly in the middle or more advanced stages of colorectal cancer that alimentary canal bleeding is seen. For a patient suffering from early colorectal cancer, the probability to be judged as positive by the fecal occult blood test is thus at a level of 50%.

Along with the progress of mass spectrometry technology in recent years, attempts have been made to diagnose specific diseases by analyzing data obtained by conducting mass spectrometry on biological samples (blood, urine, feces, saliva, or a section of biological tissues) collected from a test subject. For example, Patent Literature 1 discloses that the respective amounts of kynurenine, cystamine, 2-hydroxybutyric acid, and aspartic acid, which are in vivo metabolites contained in biological samples, are measured based on mass spectrum data obtained by the mass spectrometry on the biological samples, and the presence of early colorectal cancer in stages from 0 to 2 is determined based on the result of the measured values of those in vivo metabolites.

### CITATION LIST

### Patent Literature

Patent Literature 1: JP 2013-246080 A

### SUMMARY OF INVENTION

### Technical Problem

Colorectal cancer is grouped into stages 0 to 4 depending on the location of the cancer tissues. In colorectal cancer at stages 3 and 4, the cancer metastasizes in tissues other than those in the colon, whereas in colorectal cancer at stages 0 to 2, cancer tissues are localized to the tissues in the colon. Specifically, in colorectal cancer at stage 0, cancer tissues exist in a mucous membrane that forms the innermost layer of the colon. In colorectal cancer at stage 1, cancer tissues exist from the mucous membrane to a proper muscular layer. In colorectal cancer at stage 2, cancer tissues developing from the mucous membrane reach the vicinity of the outermost layer of the colon beyond the proper muscular layer. Taking into account such differences in the location of the cancer tissues, it is possible to expect that the kinds of in vivo metabolites contained in the biological samples change depending on stages 0 to 2. However, the inspection method disclosed in Patent Literature 1, the presence of colorectal cancer at each one of stages 0 to 2 is determined based on measurement values of the same four kinds of in vivo metabolites. Accordingly, the determination accuracy may vary depending on the stage. In addition, since the measurement values of the in vivo metabolites are derived from analysis data obtained with a gas chromatograph mass spectrometer (GC-MS), and the pretreatment for the analysis by the GC-MS is manually performed, the quantitative accuracy and reproducibility of the analysis are low. This limits the kinds of in vivo metabolites that can be quantitatively analyzed with high reproducibility. Thus, it is possible that the kinds of in vivo metabolites and their combination which allow for correct determination on the presence of early colorectal cancer are not properly chosen.

An object of the present invention is to provide a colorectal cancer inspection method by which the presence of colorectal cancer at early stages from 0 to 2 can be accurately determined.

### Solution to Problem

A colorectal cancer inspection method according to the present invention developed to solve the previously described problems includes:

measuring the amounts of at least lactic acid, pyruvic acid, and glycolic acid among a plurality of kinds of in vivo metabolites contained in a biological sample collected from a test subject, based on data obtained by performing a chromatograph-MS/MS analysis on the biological sample; and

determining the presence of colorectal cancer at any one of stages from 0 to 2, based on a measurement value of at least one of the lactic acid, the pyruvic acid, and the glycolic acid.

The “chromatograph-MS/MS analysis” is an analysis using a device including a gas or liquid chromatograph coupled with a mass spectrometer that serves as a detection device for detecting a sample separated by the gas or liquid chromatograph. Examples of the mass spectrometers include triple quadrupole mass spectrometer, Q-TOF mass spectrometer, TOF-TOF mass spectrometer, ion trap mass spectrometer, and an ion trap time-of-flight mass spectrometer. A set of data obtained by the chromatograph-MS/MS analysis is called MS/MS spectrum data. The use of the triple quadrupole mass spectrometer, Q-TOF mass spectrometer, TOF-TOF mass spectrometer, ion trap mass spectrometer, ion trap time-of-flight mass spectrometer or similar device as the detection device for detecting a sample separated by the chromatograph enables a high-sensitivity analysis even on a sample containing many impurities other than analysis targets, such as in vivo metabolites. The analysis stability is thereby improved, so that the amounts of in vivo metabolites contained in the biological sample can be quantitatively measured with high reproducibility. This allows for an accurate determination on the presence of early colorectal cancer.

The aforementioned colorectal cancer inspection method may include:

additionally measuring the amounts of ornithine and tryptophan that are in vivo metabolites contained in the biological sample; and

determining the presence of colorectal cancer at any one of stages from 0 to 2, based on measurement values of two or more kinds of in vivo metabolites including: at least one in vivo metabolite selected from the lactic acid, the pyruvic acid, and the glycolic acid; and at least one in vivo metabolite selected from the lactic acid, the pyruvic acid, the glycolic acid, the ornithine, and the tryptophan, where the latter in vivo metabolite is different from the former in vivo metabolite.

In this case, the presence of colorectal cancer at any one of stages from 0 to 2 may be determined based on the measurement values of the pyruvic acid, the glycolic acid, the ornithine, and the tryptophan.

The colorectal cancer inspection method according to the present invention may include:

additionally measuring the amounts of fumaric acid and 2-ketoisovalerate that are in vivo metabolites contained in the biological sample; and

determining the presence of colorectal cancer at any one of stages from 0 to 2, based on measurement values of the pyruvic acid, the glycolic acid, the fumaric acid, and the 2-ketoisovalerate.

The colorectal cancer inspection method according to the present invention may include:

additionally measuring the amounts of fumaric acid and malic acid that are in vivo metabolites contained in the biological sample; and

determining the presence of colorectal cancer at any one of stages from 0 to 2, based on measurement values of the lactic acid, the ornithine, the tryptophan, the fumaric acid, and the malic acid.

The colorectal cancer inspection method according to the present invention may include:

additionally measuring the amounts of fumaric acid, palmitoleic acid, lysine, and 3-hydroxyisovalerate that are in vivo metabolites contained in the biological sample; and

determining the presence of colorectal cancer at any one of stages from 0 to 2, based on measurement values of the ornithine, the glycolic acid, the tryptophan, the pyruvic acid, the fumaric acid, the palmitoleic acid, the lysine, and the 3-hydroxyisovalerate.

In one specific form of the colorectal cancer inspection method according to the present invention, the presence of colorectal cancer at stage 0 is determined based on the measurement values of the lactic acid and the ornithine.

The colorectal cancer inspection method according to the present invention may include:

additionally measuring the amounts of uric acid and glyceric acid that are in vivo metabolites contained in the biological sample, in addition to the amounts of the lactic acid, the pyruvic acid, the glycolic acid, the ornithine, and the tryptophan; and

determining the presence of colorectal cancer at stage 0, based on measurement values of the lactic acid, the glycolic acid, the uric acid, and the glyceric acid.

The colorectal cancer inspection method according to the present invention may include:

additionally measuring the amount of 2-hydroxybutyl acid that is an in vivo metabolite contained in the biological sample; and

determining the presence of colorectal cancer at stage 0, based on measurement values of the lactic acid, the 2-hydroxyutyl acid, the ornithine, and the tryptophan.

The colorectal cancer inspection method according to the present invention may include:

additionally measuring the amount of the glyceric acid that is an in vivo metabolite contained in the biological sample; and

determining the presence of colorectal cancer at stage 1, based on measurement values of the glycolic acid, the glyceric acid, and the tryptophan.

The colorectal cancer inspection method according to the present invention may include:

additionally measuring the amounts of galactose, glycine, uric acid, and glyceric acid that are in vivo metabolites contained in the biological sample; and

determining the presence of colorectal cancer at stage 1, based on measurement values of the glycolic acid, the pyruvic acid, the galactose, the glycine, the uric acid, and the glyceric acid.

The colorectal cancer inspection method according to the present invention may include:

additionally measuring the amount of citric acid that is an in vivo metabolite contained in the biological sample; and

determining the presence of colorectal cancer at stage 1, based on measurement values of the lactic acid, the ornithine, the tryptophan, and the citric acid.

The colorectal cancer inspection method according to the present invention may include:

additionally measuring the amounts of fumaric acid and saccharose that are in vivo metabolites contained in the biological sample; and

determining the presence of colorectal cancer at stage 2, based on measurement values of the pyruvic acid, the glycolic acid, the fumaric acid, and the saccharose.

The colorectal cancer inspection method according to the present invention may include:

additionally measuring the amounts of leucine, phosphoric acid, saccharose, and fumaric acid that are in vivo metabolites contained in the biological sample; and

determining the presence of colorectal cancer at stage 2, based on measurement values of the pyruvic acid, the glycolic acid, the leucine, the phosphoric acid, the saccharose, and the fumaric acid.

The colorectal cancer inspection method according to the present invention may include:

additionally measuring the amount of fumaric acid that is an in vivo metabolite contained in the biological sample; and

determining the presence of colorectal cancer at stage 2, based on measurement values of the lactic acid, the ornithine, the fumaric acid, and the tryptophan.

The present inventors collected blood from healthy subjects and colorectal cancer patients at each one of stages from 0 to 2 to search for in vivo metabolites that specifically exist in biological samples of the colorectal cancer patients at each one of stages from 0 to 2, and then used plasma components of the collected blood as samples to comprehensively measure the amounts of in vivo metabolites contained in the samples. The result revealed that certain kinds of in vivo metabolites specifically existed in samples collected from the colorectal cancer patients at the respective stages. Thus, the present invention has been conceived. Blood (plasma components) was used as the sample for conducting the search for the in vivo metabolites. A gas chromatograph mass spectrometer (GC-MS/MS) was used for analyzing the in vivo metabolites in the sample. Accordingly, it is preferable for the colorectal cancer inspection method according to the present invention to use a GC-MS/MS as the analyzing device and blood (plasma components) as the biological sample. However, the present invention is not limited thereto this combination.

Specifically, in the colorectal cancer inspection method according to the present invention, the “biological sample collected from a test subject” may be any type of sample as long as it is possible to measure the amounts of in vivo metabolites contained in the biological sample. Examples of such biological samples include blood, biological tissues, feces and urine. In view of the easiness in collection and the content of the in vivo metabolites, blood (whole blood, serum, or plasma) is preferable. In the blood, plasma is especially preferable. As the serum, a fluid element obtained by coagulating corpuscles without adding an anticoagulant agent to the whole blood can be used. As the plasma, a fluid element obtained by adding the anticoagulant agent to the whole blood so as to avoid coagulation of corpuscles can be used.

In the colorectal cancer inspection method according to the present invention, a prediction formula for distinguishing between healthy individuals and early colorectal cancer patients can be prepared by performing a multivariate analysis in which measurement values of the in vivo metabolites of the early colorectal cancer patients at each of the stages from 0 to 2 or early colorectal cancer patients at any one of stages 0 to 2 are analytically compared with those of the healthy individuals. The prediction formula is used to calculate an analysis value (p-value). If the p-value is greater than a cut-off value, it is possible to determine that the possibility of colorectal cancer is high.

As a specific example, if there are three kinds of in vivo metabolites A, B and C to be used for determining the presence of colorectal cancer at a certain stage the prediction formula for calculating the analysis value (p-value) can be expressed by the following formula (1), using the measurement values of the respective in vivo metabolites are denoted by [A], [B] and [C]:

$$P = \frac{1}{1 + {e}^{- ( i + a [ A ] + b [ B ] + c [ C ] )}} \qquad ( 1 )$$

The intercept (i) and the coefficients a to c assigned to the molecules to be analyzed (i.e., the measurement values [A], [B], and [C] of the respective in vivo metabolites) in formula (1) are determined so that the p-value calculated by formula (1) becomes closer to 0 for a healthier individual while a p-value which is closer to 1 indicates a higher probability of colorectal cancer. The p-value for each of the stages is thus calculated, and if the p-value of a certain stage is greater than the cut-off value, it is possible to determine that the probability of the colorectal cancer is high.

For evaluating the performance of the prediction formula, Receiver Operating Characteristic (ROC) analysis is used. An ROC curve obtained by an ROC analysis shows the true-positive ratio, i.e., sensitivity, on the vertical axis plotted against the false-positive ratio, i.e., “1-specificity”, on the horizontal axis. Using a cut-off value determined for the p-value, if the p-value is greater than that cut-off value, it is determined that the probability of the colorectal cancer is high; i.e., the subject tests positive. Then, the sensitivity is calculated by the ratio of the colorectal cancer patients who have tested positive to the total number of colorectal cancer patients. The false-positive ratio is also calculated by the ratio of the healthy subjects (non-disease individuals) who have tested positive for colorectal cancer positive to the total number of healthy subjects. The sensitivity and the positive ratio are further calculated in a similar manner using other cut-off values. Values thus obtained are plotted on a graph, and the ROC curve is depicted. The cut-off values can be determined based on various conditions, such as the stage of the colorectal cancer and purpose of the inspection. An analysis with the ROC curve located closer to the upper left area in the graph can be considered to be more reliable as the inspection method.

In the colorectal cancer inspection method according to the present invention, it is possible to determine the presence of colorectal cancer at each of the stages from the result of a comparison of the measurement values of the in vivo metabolites with standard values. In this case, the measurement values of the in vivo metabolites in each combination may be individually compared with the corresponding standard values, and the comparison result may be used for comprehensive judgement. The analysis result of the multiple logistic regression analysis on the measurement values of the in vivo metabolites in each combination may be compared with the standard value to determine the presence of colorectal cancer.

### Advantageous Effects of the Invention

According to the present invention, one or more kinds of in vivo metabolites contained in a biological sample are measured based on data obtained by performing a chromatograph MS/MS analysis, and the presence of colorectal cancer at any one of stages 0 to 2 is determined based on the measured values. Accordingly, the presence of early colorectal cancer can be accurately determined.

### BRIEF DESCRIPTION OF DRAWINGS

FIG. 1 is a table showing candidates of in vivo metabolites to be used for determination of the presence of colorectal cancer at each one of stages from 0 to 2 and that of a colorectal cancer at any one of stages from 0 to 2.

FIG. 2 is a table showing combinations of the in vivo metabolites to be used for prediction formulas for determination of the presence of colorectal cancer.

FIG. 3 is a graph showing an ROC curve at stage 0 in model 1.

FIG. 4 is a graph showing an ROC curve at stage 0 in model 2.

FIG. 5 is a graph showing an ROC curve at stage 0 in model 3.

FIG. 6 is a graph showing an ROC curve at stage 1 in model 1.

FIG. 7 is a graph showing an ROC curve at stage 1 in model 2.

FIG. 8 is a graph showing an ROC curve at stage 1 in model 3.

FIG. 9 is a graph showing an ROC curve at stage 2 in model 1.

FIG. 10 is a graph showing an ROC curve at stage 2 in model 2.

FIG. 11 is a graph showing an ROC curve at stage 2 in model 3.

FIG. 12 is a graph showing an ROC curve at stages 0 to 2 in model 1.

FIG. 13 is a graph showing an ROC curve at stages 0 to 2 in model 2.

FIG. 14 is a graph showing an ROC curve at stages 0 to 2 in model 3.

FIG. 15 is a graph showing an ROC curve at stages 0 to 2 in model 4.

FIG. 16 is a graph showing an ROC curve at stage 0, in which lactic acid and ornithine are used.

FIG. 17 is a graph showing an ROC curve at stage 1, in which lactic acid and ornithine are used.

FIG. 18 is a graph showing an ROC curve at stage 2, in which lactic acid and ornithine are used.

FIG. 19 is a graph showing an ROC curve at stages 0 to 2, in which lactic acid and ornithine are used.

FIG. 20 is a graph showing an ROC curve at stage 0, in which pyruvic acid and tryptophan are used.

FIG. 21 is a graph showing an ROC curve at stage 1, in which pyruvic acid and tryptophan are used.

FIG. 22 is a graph showing an ROC curve at stage 2, in which pyruvic acid and tryptophan are used.

FIG. 23 is a graph showing an ROC curve at stages 0 to 2, in which pyruvic acid and tryptophan are used.

FIG. 24 is a graph showing an ROC curve at stage 0, in which lactic acid is used.

FIG. 25 is a graph showing an ROC curve at stage 1, in which lactic acid is used.

FIG. 26 is a graph showing an ROC curve at stage 2, in which lactic acid is used.

FIG. 27 is a graph showing an ROC curve at stages 0 to 2, in which lactic acid is used.

FIG. 28 is a graph showing an ROC curve at stage 0, in which glycolic acid is used.

FIG. 29 is a graph showing an ROC curve at stage 1, in which glycolic acid is used.

FIG. 30 is a graph showing an ROC curve at stage 2, in which glycolic acid is used.

FIG. 31 is a graph showing an ROC curve at stages 0 to 2, in which glycolic acid is used.

FIG. 32 is a graph showing an ROC curve at stage 0, in which pyruvic acid is used.

FIG. 33 is a graph showing an ROC curve at stage 1, in which pyruvic acid is used.

FIG. 34 is a graph showing an ROC curve at stage 2, in which pyruvic acid is used.

FIG. 35 is a graph showing an ROC curve at stages 0 to 2, in which pyruvic acid is used.

### DESCRIPTION OF EMBODIMENTS

Hereinafter, a method of inspecting colorectal cancer according to one embodiment of the present invention is described with reference to specific examples. It should be noted that the present invention is not limited to the following embodiment.

Biological samples collected from test subjects were analyzed using a GC-MS/MS, to measure the amounts of the predetermined plural kinds of in vivo metabolites contained in the biological samples, as candidate substances. An analysis based on a multiple logistic regression analysis was conducted on the measured values (MS/MS measurement values) as the molecules to be analyzed. In this embodiment, specimens collected from colorectal cancer patients and healthy subjects were used as the biological samples, with approval by Kobe University and the medical ethics committee of National Cancer Center Japan. From the results, one or more kinds of in vivo metabolites to be used for determination of the presence of colorectal cancer at each one of stages 0 to 2 and colorectal cancer at any one of stages 0 to 2 were derived.

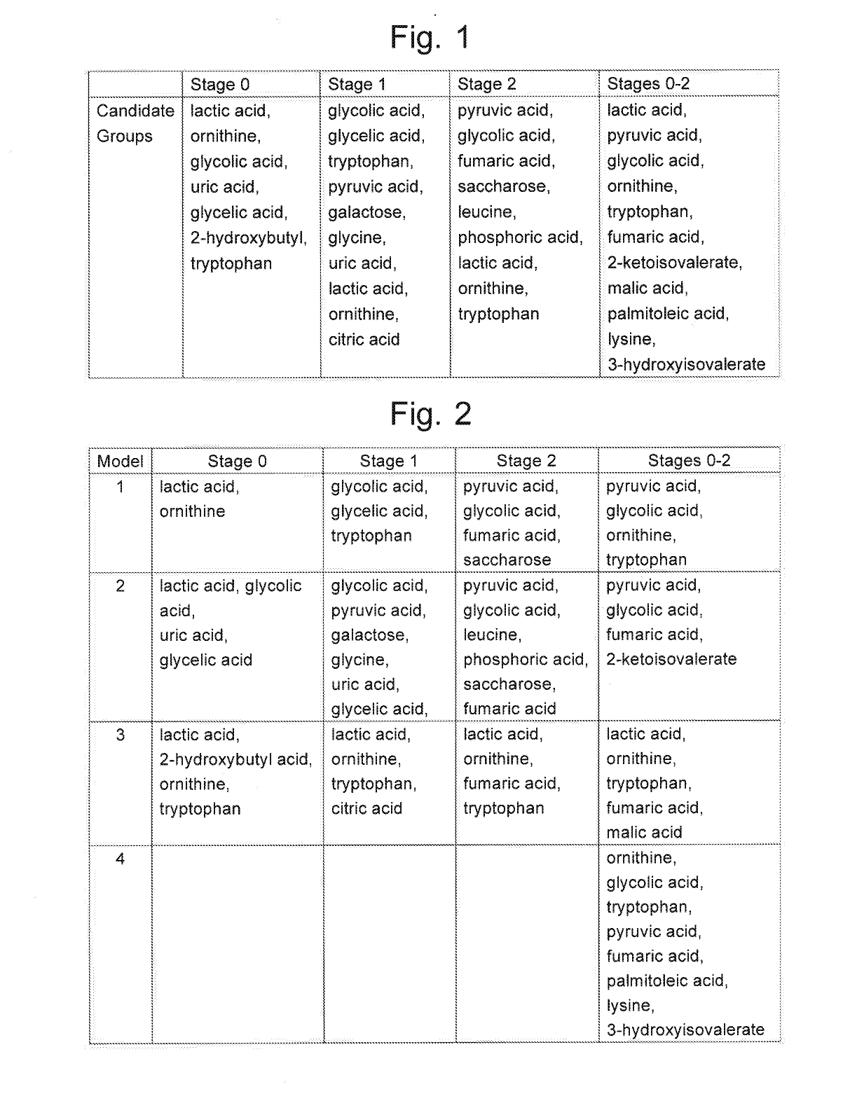

In the present embodiment, 21 kinds of in vivo metabolites were chosen as candidates of the in vivo metabolites to be used for determination of the presence of early colorectal cancer: lactic acid, ornithine, glycolic acid, uric acid, glyceric acid, 2-hydroxybutyl, tryptophan, pyruvic acid, galactose, glycine, citric acid, fumaric acid, saccharose, leucine, phosphoric acid, 2-ketoisovalerate, malic acid, palmitoleic acid, lysine, 3-hydroxyisovalerate, and aspartic acid. These 21 kinds of in vivo metabolites were sorted into four candidate groups to be respectively used for determination of the presence of the colorectal cancer at (1) stage 0, (2) stage 1, (3) stage 2 and (4) stages 0 to 2, as shown in Table of FIG. 1. From measurement results of the in vivo metabolites in each of the candidate groups, the combination of in vivo metabolites to be used for determination of the presence of colorectal cancer at the corresponding stage were derived. Here, the selection of 21 kinds of in vivo metabolites and their sorting into the four candidate groups were conducted with reference to information relating to known colorectal cancer markers, such as substances previously known as the colorectal cancer markers and substances which are likely to be available as such markers.

### <Preparation of Samples>

Blood was collected from the aforementioned colorectal cancer patients and healthy subjects, who were in a food-deprived state, into a blood-collecting vessel containing ethylenediaminetetraacetic acid (EDTA), to be mixed with the EDTA. The blood was then stored at 4° C. followed by undergoing centrifugation (3,000 rpm, at 4° C. and for ten minutes), to thereby obtain plasma.

Then, 50 μL of the obtained plasma was dispensed into a tube, and 270 μL of methanol containing 2-isopropylmalic acid and a stable isotope reagent of the in vivo metabolites as the internal standard was added to and mixed with the plasma.

Subsequently, moisture was removed from the mixed solution by a freeze dryer, and then methylhydroxyamine hydrochloride dissolved in pyridine was added. The obtained result was shaken at 30° C. for 90 minutes for oxime-derivatization. Then, N-Methyl-N-(trimethylsilyl)trifluoroacetamide (MSTFA) was added thereto, and the mixture was shaken at 37° C. for 30 minutes for trimethylsilylation. The obtained result was used as a sample.

### <Collection of Mass Spectrum Data (MS/MS Data)>

For each of those samples, mass spectrometry was conducted using a triple quadrupole gas chromatograph-mass spectrometer (GCMS-TQ8040, manufactured by Shimadzu Corporation), to collect MS/MS data. The mass spectrometry was conducted under the following conditions.

The injection volume of the sample was set to 1 μL, and BPX-5 (manufactured by SGE Analytical Science Pty. Ltd) was used as a GC capillary column. The temperature of the column was kept at 60° C. for 2 minutes from the initiation of the measurement. Then, the temperature was increased by 15° C. per minute, up to 330° C. and was kept at 330° C. for three minutes. The temperature of the ion source was set to 250° C.

### <Measurement of In Vivo Metabolites>

Peaks on the mass spectrum were comprehensively detected from the MS/MS data of each sample. The peak information (mass-to-charge ratio and signal intensity) of the detected peaks, and the mass-to-charge ratios of substances specific to multiple in vivo metabolites stored in an MS library, the peak information of the internal standard, and other related information were used to obtain measurement values (hereinafter, referred to as an “MS/MS measurement value”) of each of these in vivo metabolites.

### EXAMPLE 1

The MS/MS measurement values of the in vivo metabolites collected from more than 70 samples of the healthy subjects and colorectal cancer patients were analyzed by an appropriate data-analyzing method, and then the presence of colorectal cancer at each one of stages 0 to 2 and that of colorectal cancer at any one of stages 0 to 2 were determined. In Example 1, one or more in vivo metabolites to be used for the determination were chosen, and prediction formulas were prepared using the MS/MS measurement values of these in vivo metabolites. The prediction formulas were used to determine the presence of colorectal cancer. In Example 1, a formula for calculating an analysis value (p-value) with the multiple logistic regression analysis on the MS/MS measurement values of the in vivo metabolites was used as the prediction formula. The p-value calculated by this prediction formula becomes closer to 0 for a healthier individual. Accordingly, it is possible to determine that colorectal cancer is more likely to be present as the p-value becomes closer to 1. FIG. 2 is a table showing four models of the combinations of the in vivo metabolites used for the prediction formulas for determination of the presence of colorectal cancer.

For the model 1 shown in FIG. 2, the MS/MS measurement values of lactic acid and ornithine were used to prepare a prediction formula for the colorectal cancer at stage 0, by the multiple logistic regression analysis method. The MS/MS measurement values of glycolic acid, glyceric acid, and tryptophan were used to prepare a prediction formula for the colorectal cancer at stage 1. The MS/MS measurement values of pyruvic acid, glycolic acid, fumaric acid, and succharose were used to prepare a prediction formula for the colorectal cancer at stage 2. The MS/MS measurement values of ornithine, glycolic acid, tryptophan, and pyruvic acid were used to prepare a prediction formula for the colorectal cancer at any one of stages from 0 to 2.

For the model 2 shown in FIG. 2, the measurement values of lactic acid, glycolic acid, uric acid, and glyceric acid were used to prepare a prediction formula for the colorectal cancer at stage 0, by a stepwise method. The measurement values of glycolic acid, uric acid, glyceric acid, pyruvic acid, galactose, and glycine were used to prepare a prediction formula for the colorectal cancer at stage 1. The measurement values of glycolic acid, pyruvic acid, leucine, phosphoric acid, succharose, and fumaric acid were used to prepare a prediction formula for the colorectal cancer at stage 2. The MS/MS measurements values of glycolic acid, pyruvic acid, fumaric acid, and 2-ketoisovalerate were used to prepare a prediction formula for the colorectal cancer at any one of stages from 0 to 2.

For the model 3 shown in FIG. 2, in vivo metabolites whose measurement values could be corrected by a stable isotope were preferentially selected from among each of the candidate groups shown in FIG. 1. The MS/MS measurement values of lactic acid, 2-hydroxybutyric acid, ornithine, and tryptophan were used to prepare a prediction formula for the colorectal cancer at stage 0, by the stepwise method. The MS/MS measurement values of lactic acid, ornithine, tryptophan, and citric acid were used to prepare a prediction formula for the colorectal cancer at stage 1, by the multiple logistic regression analysis method. The MS/MS measurement values of lactic acid, ornithine, tryptophan, and fumaric acid were used to prepare a prediction formula for the colorectal cancer at stage 2, by the multiple logistic regression analysis method. The MS/MS measurement values of lactic acid, ornithine, tryptophan, fumaric acid, and malic acid were used to prepare a prediction formula for the colorectal cancer at any one of stages from 0 to 2, by the multiple logistic regression analysis method.

For the model 4 shown in FIG. 2, many in vivo metabolites selected from the candidate groups shown in FIG. 1 were combined to prepare a more sophisticated prediction formula. Then, the MS/MS measurement values of ornithine, glycolic acid, tryptophan, pyruvic acid, fumaric acid, palmitoleic acid, lysine, and 3-hydroxyisovalerate were used to prepare a prediction formula for colorectal cancer at any one of stages from 0 to 2, by the multiple logistic regression analysis method.

The determination results based on the prediction formulas prepared by using the MS/MS measurement values of the in vivo metabolites in models 1 to 4 are hereinafter described.

### <1. Determination of the Presence of Colorectal Cancer at Stage 0>

For 79 healthy subjects and 79 colorectal cancer patients at stage 0, with each group having similar compositions in age, gender and BMI, the MS/MS measurement values of the in vivo metabolites indicated in models 1 to 3 shown in FIG. 2 were calculated based on the before-mentioned methods. The obtained MS/MS measurement values were used to establish the following formulas 2 to 4. Formulas 2, 3, and 4 respectively express the prediction formulas using the in vivo metabolites of models 1, 2, and 3. The respective descriptions of [lactic acid], [ornithine], and so on in the formulas indicate the respective MS/MS measurement values of the in vivo metabolites indicated in parentheses.

P=1/[1+exp{−(−13.506931+49.9697673*[lactic acid]+4.26878539*[ornithine])}] Formula 2

P=1/[1+exp{−(−27.2172815+97.973150029*[lactic acid]+219.03142781*[glycolic acid]−5.222210335*[uric acid]−68.8976813*[glyceric acid])}] Formula 3

P=1/[1+exp{−(−5.945008762+61.715859951*[lactic acid]−0.690292331*[2−hydroxybutyric acid]+7.782649017*[ornithine]−9.877482547*[tryptophan])}] Formula 4

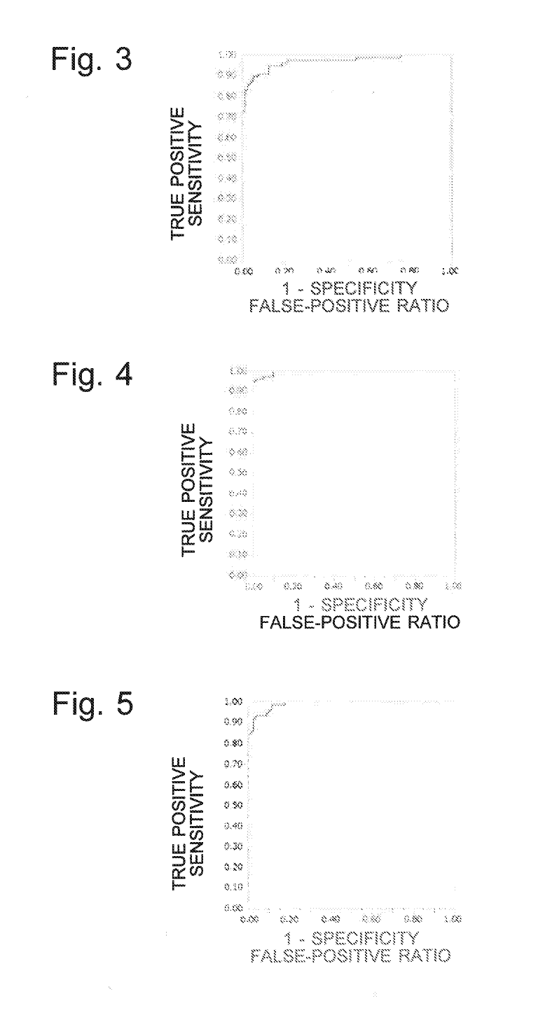

FIGS. 3 to 5 show the results of ROC analyses in which the cut-off values of the p-values obtained using the before-mentioned formulas 2, 3, and 4 were respectively set to 0.63, 0.6, and 0.5. FIGS. 3, 4, and 5 respectively show the analysis results (ROC curves) of the models 1, 2, and 3. Models 1, 2, and 3 respectively have sensitivities of 89.9, 96.2, and 92.4%, specificities of 94.9, 98.7, and 97.5%, and proper diagnosis ratios of 92.4, 97.5, and 95.0%.

### <2. Determination of the Presence of Colorectal Cancer at Stage 1>

For 80 healthy subjects and 80 colorectal cancer patients at stage 1, with each group having similar compositions in age, gender and BMI, the MS/MS measurement values of the in vivo metabolites indicated in models 1 to 3 shown in FIG. 2 were calculated using the same methods as mentioned earlier in the case of stage 0. The obtained MS/MS measurement values were used to establish the following formulas 5 to 7. Formulas 5, 6, and 7 respectively express the prediction formulas using the combinations of the in vivo metabolites in models 1, 2, and 3.

P=1/(1+exp(−(−5.244098866−39.34118597*[glyceric acid]+137.16175377*[glycolic acid]−3.169957639*[tryptophan]))) Formula 5

P=1/(1+exp(−(−14.99143949+373.05777093*[glycolic acid]+36.504337562*[pyruvic acid]−1.92165145*[galactose]−198.2189077*[glycine]−3.311584205*[uric acid]−30.15525187*[glyceric acid]))) Formula 6

P=1/(1+exp(−(3.5655473738+25.015349738*[lactic acid]+4.7551912842*[ornithine]−7.667356818*[tryptophan]−14.85882644*[citric acid]))) Formula 7

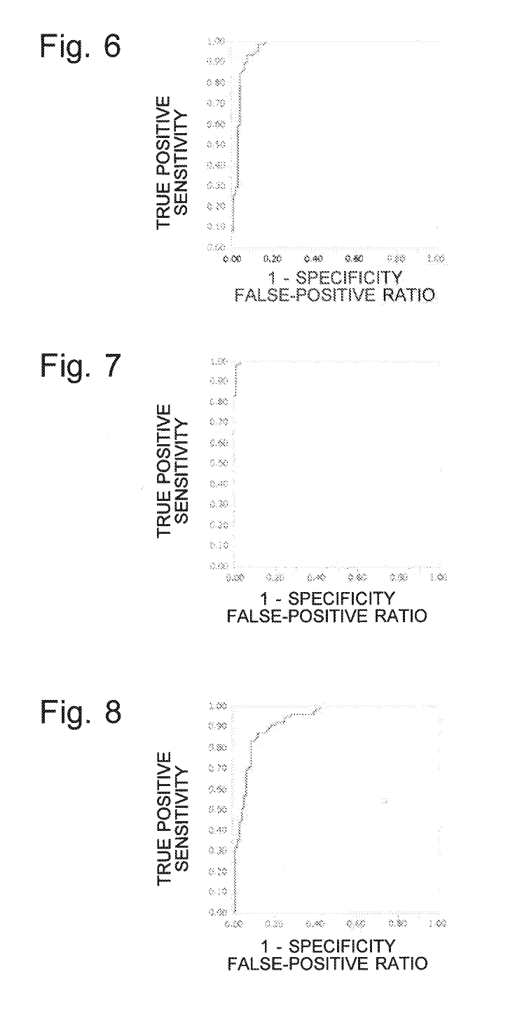

FIGS. 6 to 8 show the results of ROC analyses in which the cut-off values of the p-values obtained from the before-mentioned formulas 5, 6, and 7 were respectively set to 0.48, 0.495, and 0.489. FIGS. 6, 7, and 8 respectively show the analysis results (ROC curves) of models 1, 2, and 3. The models 1, 2, and 3 respectively have the sensitivities of 93.8, 98.8, and 87.5%, specificities of 92.4, 98.9, and 88.0%, and proper diagnosis ratios of 93.1, 98.8, and 87.8%.

### <3. Determination of the Presence of Colorectal Cancer at Stage 2>

For 123 healthy subjects and 123 colorectal cancer patients at the stage 2, with each group having similar compositions in age, gender and BMI, the MS/MS measurement values of the in vivo metabolites indicated in models 1 to 3 shown in FIG. 2 were calculated using the same methods as mentioned earlier in the case of stage 0. The obtained MS/MS measurement values were used to establish the following formulas 8 to 10. Formulas 8, 9, and 10 respectively express the prediction formulas using the combinations of the in vivo metabolites in models 1, 2, and 3.

P=1/(1+exp(−(−19.5140397+22.817689838*[pyruvic acid]+68.301755498*[glycolic acid]+214.36183256*[fumaric acid]+25.33111413*[succharose]))) Formula 8

P=1/(1+exp(−(−13.29001781+41.289331856*[pyruvic acid]+147.27766415*[glycolic acid]−1.508033407*[leucine]−3.04399115*[phosphoric acid]+82.577937486*[succharose]+361.20773121*[fumaric acid]))) Formula 9

P=1/(1+exp(−(−4.98049072+25.353666196*[lactic acid]+7.0154960778*[ornithine]+118.40574539*[fumaric acid]−6.242588904[tryptophan]))) Formula 10

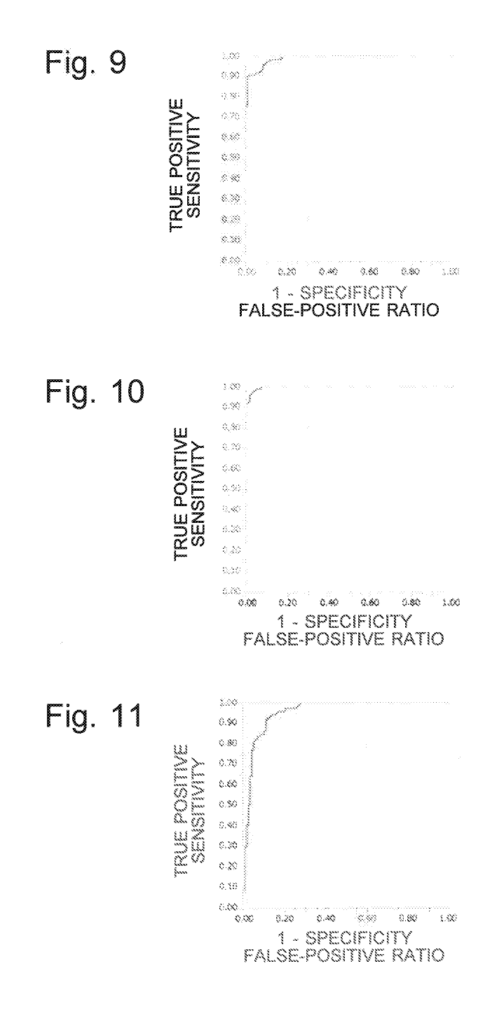

FIGS. 9 to 11 show the results of ROC analyses in which the cut-off values of the p-values obtained using the before-mentioned Formulas 8, 9, and 10 were respectively set to 0.75, 0.5, and 0.41. FIGS. 9, 10, and 11 respectively show the analysis results (ROC curves) of models 1, 2, and 3. The models 1, 2, and 3 respectively have the sensitivities of 90.2, 96.8, and 91.9%, specificities of 99.2, 98.3, and 89.2%, and proper diagnosis ratios of 94.7, 97.6, and 90.6%.

### <4. Determination of the Presence of Colorectal Cancer at Any One of Stages 0 to 2>

For 282 healthy subjects and 282 colorectal cancer patients at any one of stages 0 to 2, with each group having similar compositions in age, gender and BMI, the MS/MS measurement values of the in vivo metabolites indicated by models 1 to 3 shown in FIG. 2 were calculated using the same methods as mentioned earlier in the cases of stages 0 to 2. The obtained MS/MS measurement values were used to establish the following formulas 11 to 13. Formulas 11, 12, 13, and 14 respectively express the prediction formulas using the combinations of the in vivo metabolites in models 1, 2, 3, and 4.

P=1/(1+exp(−(−8.910098221+16.291903021*[pyruvic acid]+68.3503626855752*[glycolic acid]−7.011317019*[tryptophan]+4.9721464301*[ornithine]))) Formula 11

P=1/(1+exp(−ketoisovalerate(−11.00126848+23.045661795*[pyruvic acid]+62.493803471*[glycolic acid]−583.6163492*[2-ketoisovalerate]+235.04693604*[fumaric acid]))) Formula 12

P=1/(1+exp(−(−3.340652437+29.789478677*[lactic acid]−7.212093514*[tryptophan]+5.5597551347*[ornithine]+237.87196106*[fumaric acid]−59.94751789*[malic acid]))) Formula 13

P=1/(1+exp(−(−8.992407686+19.375560943*[pyruvic acid]+82.332483986*[glycolic acid]−6.864158669*[tryptophan]+9.6859550708*[ornithine]−67.47807685*[palmitoleic acid]−5.757937815*[lysine]−24.99353052*[3−hydroxyisovalerate]+296.32483475*[fumaric acid]))) Formula 14

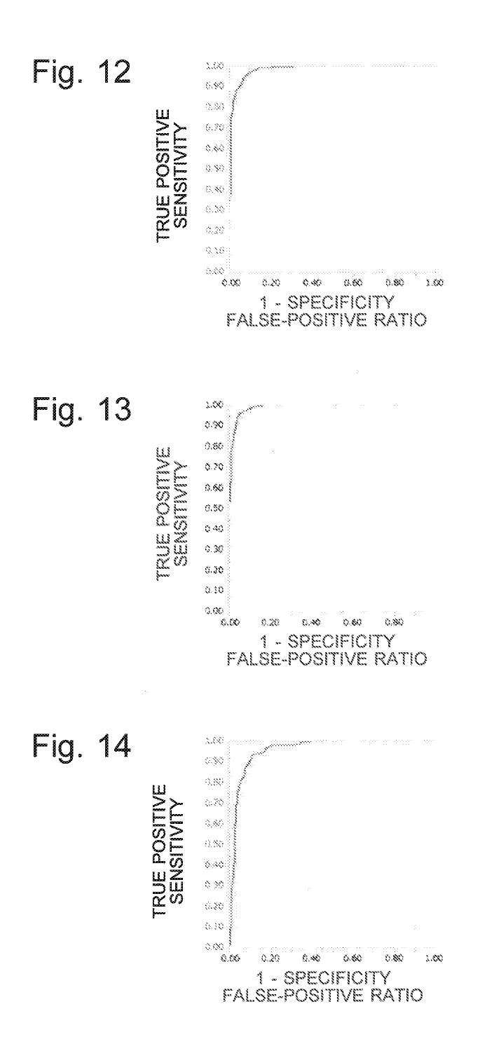

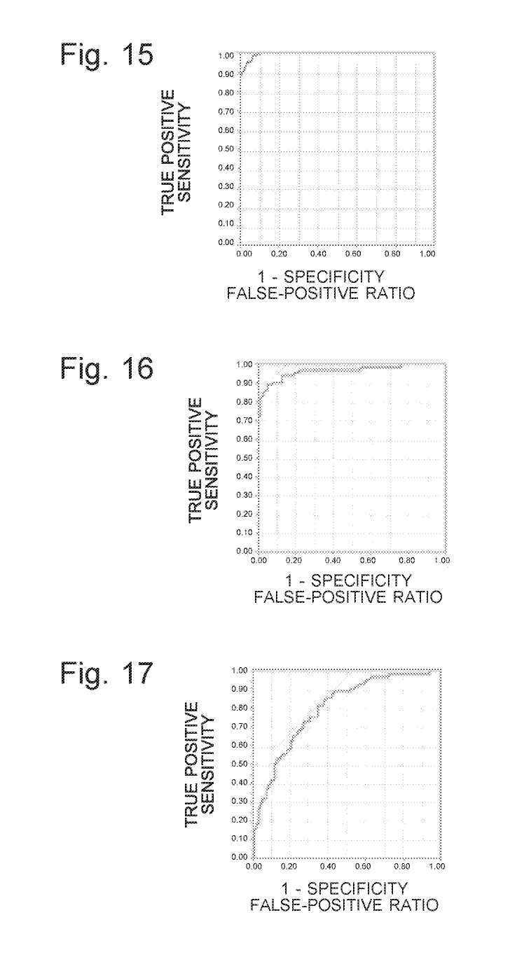

FIGS. 12 to 15 show the results of ROC analyses in which the cut-off values of the p-values obtained using the before-mentioned Formulas 11, 12, 13, and 14 were respectively set to 0.45, 0.45, 0.4, and 0.19. FIGS. 12, 13, 14, and 15 respectively show the analysis results (ROC curves) of models 1, 2, 3, and 4. The models 1, 2, 3, and 4 respectively have the sensitivities of 95.4, 96.5, 93.3, and 98.3%, specificities of 92.8, 94.9, 89.7, and 93.8%, and proper diagnosis ratios of 94.1, 95.7, 91.5, and 96.1%.

The following Table 1 indicates altogether the results of the determination of the presence of colorectal cancers at each of the stages based on the p-values calculated by the prediction formulas of the aforementioned models 1 to 4. In Table 1, “AUC” indicates area under the blood concentration-time curve.

TABLE 1

| | | | | | | Proper |
|---|---|---|---|---|---|---|
| | | | Cut-off | Sensitivity | Specificity | diagnosis |
| Stage | Model | AUC | value | (%) | (%) | ratio (%) |
| 0 | 1 | 0.96892 | 0.63 | 89.9 | 94.9 | 92.4 |
| | 2 | 0.99664 | 0.6 | 96.2 | 98.7 | 97.5 |
| | 3 | 0.99023 | 0.5 | 92.4 | 97.5 | 95.0 |
| 1 | 1 | 0.96236 | 0.48 | 93.8 | 92.4 | 93.1 |
| | 2 | 0.99796 | 0.495 | 98.8 | 98.9 | 98.8 |
| | 3 | 0.9284 | 0.489 | 87.5 | 88.0 | 87.8 |
| 2 | 1 | 0.98977 | 0.75 | 90.2 | 99.2 | 94.7 |
| | 2 | 0.99776 | 0.5 | 96.8 | 98.3 | 97.6 |
| | 3 | 0.9565 | 0.41 | 91.9 | 89.2 | 90.6 |
| 0 to 2 | 1 | 0.98688 | 0.45 | 95.4 | 92.8 | 94.1 |
| | 2 | 0.9906 | 0.45 | 96.5 | 94.9 | 95.7 |
| | 3 | 0.95981 | 0.4 | 93.3 | 89.7 | 91.5 |
| | 4 | 0.99636 | 0.19 | 98.3 | 93.8 | 96.1 |

As can be understood from Table 1, it was suggested that early colorectal cancer at stages 0 to 2 could be accurately diagnosed with high sensitivity for each one of these stages, by any of the prediction formulas of the models 1 to 4. It was also suggested that early colorectal at any one of stages 0 to 2 could be accurately diagnosed with high sensitivity.

### EXAMPLE 2

In Example 2, for 282 healthy subjects and 282 colorectal cancer patients, with each group having similar compositions in age, gender and BMI, the MS/MS measurement values of the in vivo metabolites were calculated using the same methods as used in Example 1. For the obtained MS/MS measurement values, an appropriate data analysis was conducted, to determine the presence of colorectal cancer at each one of stages 0 to 2, or colorectal cancer at any one of stages 0 to 2, by methods different from those used in Example 1. As such, Example 2 is hereinafter described.

Example 2 differs from Example 1 in that the presence of the colorectal cancer at each one of stages 0 to 2, and that of the colorectal cancer at any one of stages 0 to 2 were determined using the MS/MS measurement values of the same in vivo metabolites.

### <1. Determination Using Lactic Acid and Ornithine>

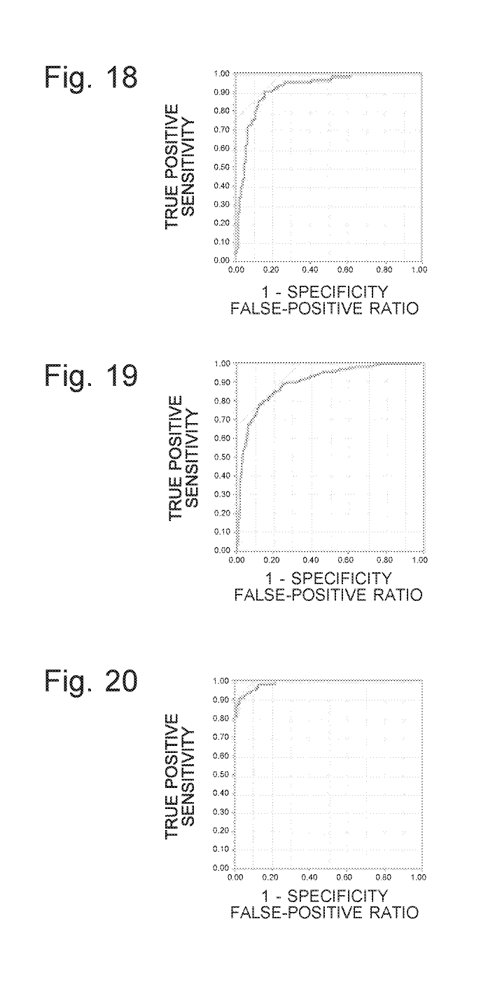

The prediction formulas for the respective stages using the MS/MS measurement values of lactic acid and ornithine are shown below. FIGS. 16 to 19, and Table 2 below show the cut-off values of p-values obtained from each of the prediction formulas and the results of ROC analyses performed using those cut-off values.

P=1/[1+exp{−(−13.506931+49.9697673*[lactic acid]+4.26878539*[ornithine])}] <Stage 0>

P=1/[1+exp{−(−3.87236783+15.326042355*[lactic acid]+1.2883608996*[ornithine])}] <Stage 1>

P=1/[1+exp{−(−8.659104135+25.441015108*[lactic acid]+5.2519152847*[ornithine])}] <Stage 2>

P=1/[1+exp{−(−7.109178221+24.600621297*[lactic acid]+3.2254288676*[ornithine])}] <Stages 0 to 2>

TABLE 2

| | | Stage | | | |
|---|---|---|---|---|---|
| Model | | 0 | I | II | 0-II |
| Lactic acid, and | AUC | 0.96892 | 0.80245 | 0.92364 | 0.89782 |
| Ornithine | Cut-off value | 0.630 | 0.367 | 0.370 | 0.483 |
| | Sensitivity (%) | 89.9 | 82.5 | 91.9 | 80.1 |
| | Specificity (%) | 94.9 | 65.2 | 84.2 | 85.9 |
| | Proper | 92.4 | 73.4 | 88.0 | 83.0 |
| | diagnosis ratio (%) | | | | |
| Pyruvic acid, | AUC | 0.98991 | 0.89158 | 0.9689 | 0.95227 |
| Tryptophan | Cut-off value | 0.6000 | 0.4120 | 0.5930 | 0.4190 |
| | Sensitivity (%) | 92.4 | 85.0 | 88.6 | 90.1 |
| | Specificity (%) | 97.5 | 84.5 | 95.0 | 90.4 |
| | Proper | 95.0 | 84.9 | 91.8 | 90.3 |
| | diagnosis ratio (%) | | | | |
| Lactic acid | AUC | 0.95944 | 0.81033 | 0.9 | 0.88963 |
| | Cut-off value | 0.2060 | 0.1745 | 0.1952 | 0.1952 |
| | Sensitivity (%) | 93.7 | 80.0 | 87.0 | 82.6 |
| | Specificity (%) | 91.1 | 73.9 | 84.2 | 82.8 |
| | Proper | 92.2 | 77.0 | 85.6 | 82.7 |
| | diagnosis ratio (%) | | | | |
| Glycolic acid | AUC | 0.94552 | 0.87514 | 0.89614 | 0.90352 |
| | Cut-off value | 0.125731 | 0.1115 | 0.1195 | 0.1147 |
| | Sensitivity (%) | 89.9 | 86.3 | 97.6 | 92.6 |
| | Specificity (%) | 93.7 | 79.4 | 77.5 | 78.0 |
| | Proper | 91.7 | 82.9 | 87.6 | 85.3 |
| | diagnosis ratio (%) | | | | |
| Pyruvic acid | AUC | 0.98654 | 0.84891 | 0.95950 | 0.93551 |
| | Cut-off value | 0.2715 | 0.2800 | 0.3140 | 0.2930 |
| | Sensitivity (%) | 97.5 | 77.5 | 91.9 | 87.9 |
| | Specificity (%) | 89.9 | 88.0 | 93.3 | 90.4 |
| | Proper | 93.7 | 82.8 | 92.6 | 89.2 |
| | diagnosis ratio | | | | |
| | (%) | | | | |

### <2. Determination Using Pyruvic Acid and Tryptophan>

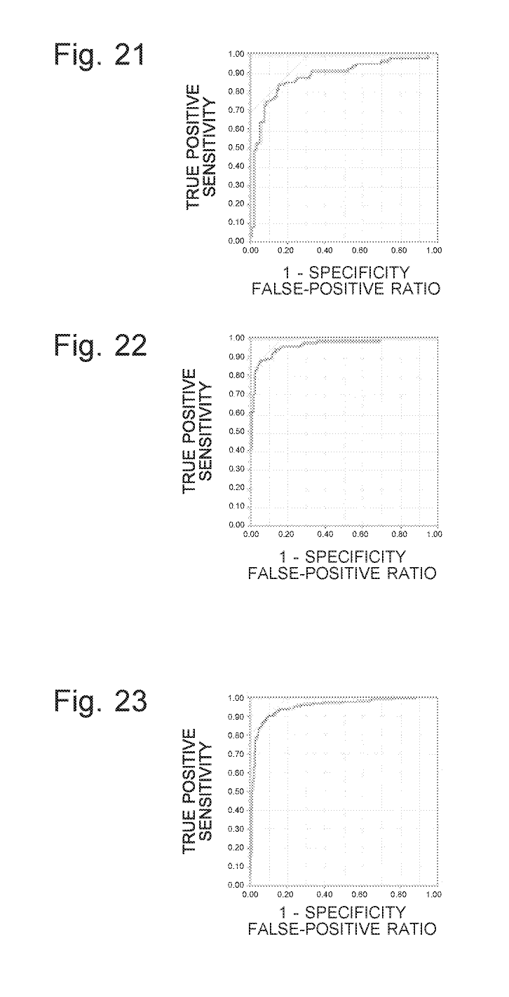

The prediction formulas for the respective stages using the MS/MS measurement values of lactic acid and ornithine are hereinafter shown. FIGS. 20 to 23, and Table 2 above show the cut-off values of the p-values obtained from each of the prediction formulas and the results of ROC analyses performed using those cut-off values.

P=1/[1+exp{−(−3.024371454−4.885245141*[tryptophan]+29.99823801*[pyruvic acid])}] <Stage 0>

P=1/[1+exp{−(1.4528990406−4.070618917*[tryptophan]+9.2766738843*[pyruvic acid])}] <Stage 1>

P=1/[1+exp{−(−0.438767321−4.66996093*[tryptophan]+17.635049962*[pyruvic acid])}] <Stage 2>

P=1/[1+exp{−(−0.20906188−3 .903240175*[tryptophan]+14.960074677*[pyruvic acid])}] <Stages 0 to 2>

### <3. Determination Using Lactic Acid>

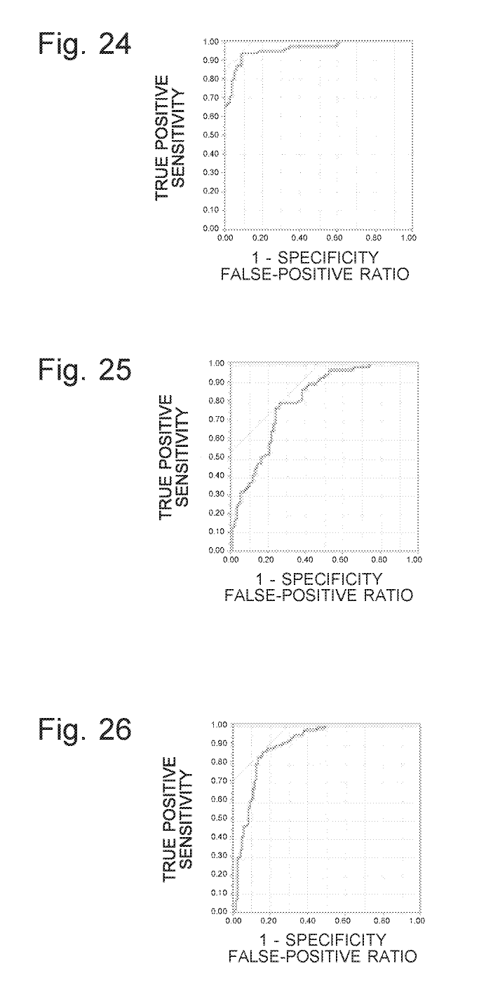

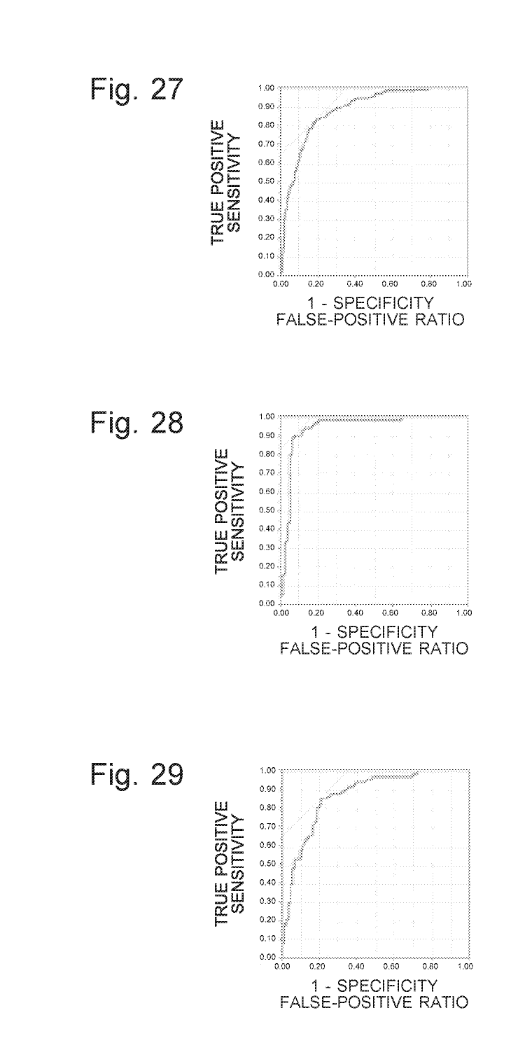

The presence of colorectal cancer at each one of stages 0 to 2 and that of the colorectal cancer at any one of stages 0 to 2 were determined using only the MS/MS measurement value of lactic acid. The results are shown in Table 2. Like this example, no prediction formula was prepared when the MS/MS measurement value of a single in vivo metabolite was used. When the MS/MS measurement value was greater than the cut-off value, the probability of the colorectal cancer was judged high (positive). Then, the sensitivity, specificity, and proper diagnosis ratio were calculated based on the determination results. FIGS. 24 to 27 each show the ROC curve prepared from thus obtained sensitivity and specificity.

### <4. Determination Using Glycolic Acid>

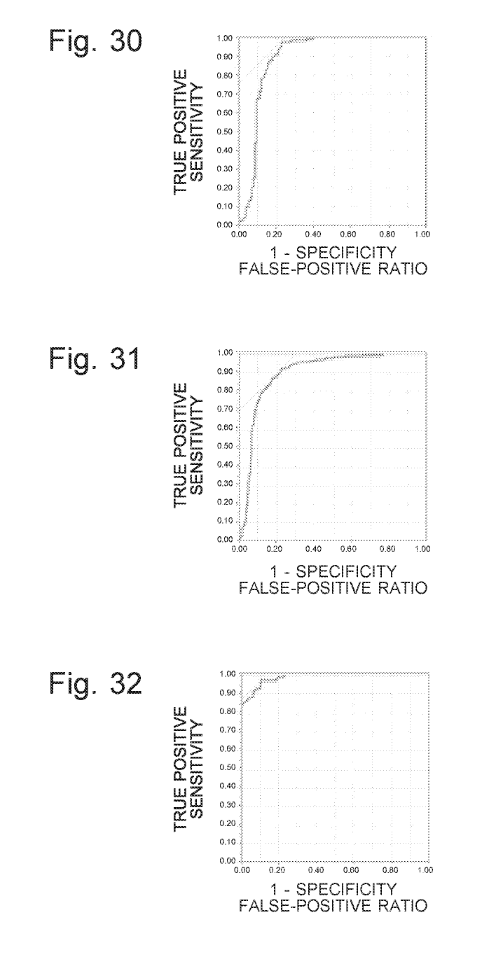

The presence of colorectal cancer at each one of stages 0 to 2 and that of the colorectal cancer at any one of stages 0 to 2 were determined using only the MS/MS measurement value of glycolic acid. The results are shown in Table 2. In this example, the prediction formula was also not prepared, just as in the determination using the lactic acid. When the MS/MS measurement value was greater than the cut-off value, the probability of the colorectal cancer was judged high (positive). Then, the sensitivity, specificity, and proper diagnosis ratio were calculated based on the determination results. FIGS. 28 to 31 each show the ROC curve prepared from thus obtained sensitivity and specificity.

### <5. Determination Using Pyruvic Acid>

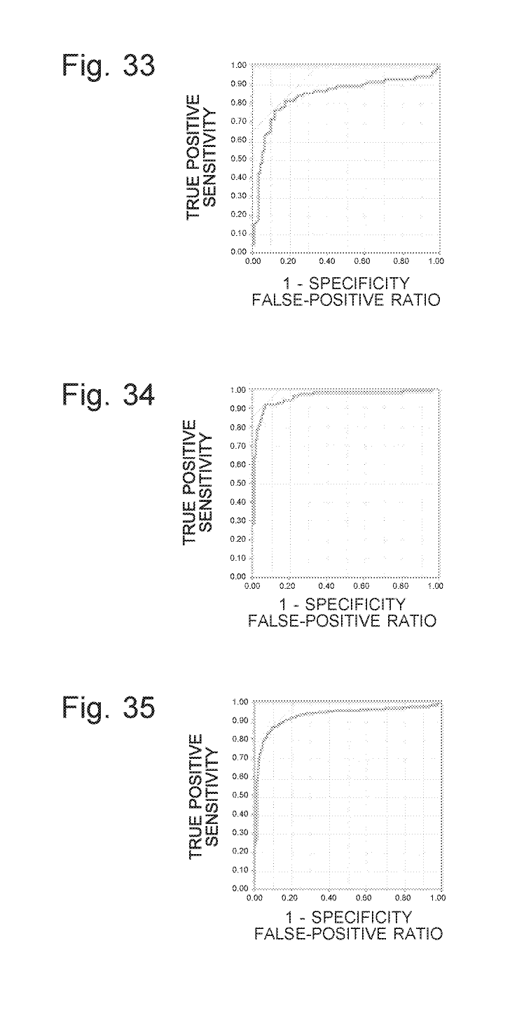

The presence of colorectal cancer at each one of stages 0 to 2 and that of the colorectal cancer at any one of stages 0 to 2 were determined using only the MS/MS measurement value of pyruvic acid. The results are shown in Table 2. In this example, the prediction formula was also not prepared, just as in the determination using the lactic acid. When the MS/MS measurement value was greater than the cut-off value, the probability of the colorectal cancer was judged high (positive). Then, the sensitivity, specificity, and proper diagnosis ratio were calculated based on the determination results. FIGS. 32 to 35 each show the ROC curve prepared from thus obtained sensitivity and specificity.

As can be understood from Table 2, it was suggested that the colorectal cancer at all of the stages 0 to 2 could be accurately diagnosed with high sensitivity, even when the MS/MS measurement values of one or more in vivo metabolites were commonly used.

Especially, the proper diagnosis ratio of 80% or more was obtained at all stages by the determination of the presence of colorectal cancer using (i) pyruvic acid and tryptophan, (ii) only glycolic acid, and (iii) only pyruvic acid. Accordingly, it could be presumed that the MS/MS measurement values of these in vivo metabolites could be effective markers for colorectal cancer.

1. A colorectal cancer inspection method, comprising:
measuring amounts of at least lactic acid, pyruvic acid, and glycolic acid among a plurality of kinds of in vivo metabolites contained in a biological sample collected from a test subject, based on data obtained by performing a chromatograph-MS/MS analysis on the biological sample; and
determining presence of colorectal cancer at any one of stages from 0 to 2, based on a measurement value of at least one of the lactic acid, the pyruvic acid, and the glycolic acid.

2. The colorectal cancer inspection method according to claim 1, comprising:
additionally measuring amounts of ornithine and tryptophan that are in vivo metabolites contained in the biological sample; and
determining the presence of colorectal cancer at any one of stages from 0 to 2, based on measurement values of two or more kinds of in vivo metabolites including: at least one in vivo metabolite selected as a first metabolite from the lactic acid, the pyruvic acid, and the glycolic acid; and at least one in vivo metabolite selected as a second metabolite from the lactic acid, the pyruvic acid, the glycolic acid, the ornithine, and the tryptophan, where the second in vivo metabolite is different from the first in vivo metabolite.

3. The colorectal cancer inspection method according to claim 2, wherein the presence of colorectal cancer at any one of stages from 0 to 2 is determined based on the measurement values of the pyruvic acid, the glycolic acid, the ornithine, and the tryptophan

4. The colorectal cancer inspection method according to claim 2, comprising:
additionally measuring amounts of fumaric acid and 2-ketoisovalerate that are in vivo metabolites contained in the biological sample; and
determining the presence of colorectal cancer at any one of stages from 0 to 2, based on measurement values of the pyruvic acid, the glycolic acid, the fumaric acid, and the 2-ketoisovalerate.

5. The colorectal cancer inspection method according to claim 2, comprising:
additionally measuring amounts of fumaric acid and malic acid that are in vivo metabolites contained in the biological sample; and
determining the presence of colorectal cancer at any one of stages from 0 to 2, based on measurement values of the lactic acid, the ornithine, the tryptophan, the fumaric acid, and the malic acid.

6. The colorectal cancer inspection method according to claim 2, comprising: additionally measuring amounts of fumaric acid, palmitoleic acid, lysine, and 3-hydroxyisovalerate that are in vivo metabolites contained in the biological sample; and
determining the presence of colorectal cancer at any one of stages from 0 to 2, based on measurement values of the ornithine, the glycolic acid, the tryptophan, the pyruvic acid, the fumaric acid, the palmitoleic acid, the lysine, and the 3-hydroxyisovalerate.

7. The colorectal cancer inspection method according to claim 2, wherein the presence of colorectal cancer at stage 0 is determined based on the measurement values of the lactic acid and the ornithine.

8. The colorectal cancer inspection method according to claim 2, comprising:
additionally measuring amounts of uric acid and glyceric acid that are in vivo metabolites contained in the biological sample; and
determining the presence of colorectal cancer at stage 0, based on measurement values of the lactic acid, the glycolic acid, the uric acid, and the glyceric acid.

9. The colorectal cancer inspection method according to claim 2, comprising:
additionally measuring an amount of 2-hydroxybutyl acid that is an in vivo metabolite contained in the biological sample; and
determining the presence of colorectal cancer at stage 0, based on measurement values of the lactic acid, the 2-hydroxyutyl acid, the ornithine, and the tryptophan.

10. The colorectal cancer inspection method according to claim 2, comprising:
additionally measuring an amount of the glyceric acid that is an in vivo metabolite contained in the biological sample; and
determining the presence of colorectal cancer at stage 1, based on measurement values of the glycolic acid, the glyceric acid, and the tryptophan.

11. The colorectal cancer inspection method according to claim 2, comprising:
additionally measuring amounts of galactose, glycine, uric acid, and glyceric acid that are in vivo metabolites contained in the biological sample; and
determining the presence of colorectal cancer at stage 1, based on measurement values of the glycolic acid, the pyruvic acid, the galactose, the glycine, the uric acid, and the glyceric acid.

12. The colorectal cancer inspection method according to claim 2, comprising:
additionally measuring an amount of citric acid that is an in vivo metabolite contained in the biological sample; and
determining the presence of colorectal cancer at stage 1, based on measurement values of the lactic acid, the ornithine, the tryptophan, and the citric acid.

13. The colorectal cancer inspection method according to claim 2, comprising:
additionally measuring amounts of fumaric acid and saccharose that are in vivo metabolites contained in the biological sample; and
determining the presence of colorectal cancer at stage 2, based on measurement values of the pyruvic acid, the glycolic acid, the fumaric acid, and the saccharose.

14. The colorectal cancer inspection method according to claim 2, comprising:
additionally measuring the amounts of leucine, phosphoric acid, saccharose, and fumaric acid that are in vivo metabolites contained in the biological sample; and
determining the presence of colorectal cancer at stage 2, based on measurement values of the pyruvic acid, the glycolic acid, the leucine, the phosphoric acid, the saccharose, and the fumaric acid.

15. The colorectal cancer inspection method according to claim 2, comprising:
additionally measuring an amount of fumaric acid that is an in vivo metabolite contained in the biological sample; and
determining the presence of colorectal cancer at stage 2, based on measurement values of the lactic acid, the ornithine, the fumaric acid, and the tryptophan.

16. The colorectal cancer inspection method according to claim 1, wherein the biological sample is one selected from whole blood, plasma, and serum.

17. The colorectal cancer inspection method according to claim 16, wherein the biological sample is the plasma.

18. The colorectal cancer inspection method according to claim 1, wherein the chromatograph-MS/MS analysis is a gas chromatograph-MS/MS analysis.

19. The colorectal cancer inspection method according to claim 1, wherein the presence of colorectal cancer at each of the stages is determined from an analysis result obtained by a multiple logistic regression analysis on the measurement value of each of the in vivo metabolites.

20. The colorectal cancer inspection method according to claim 19, wherein it is determined that the colorectal cancer is likely to be present when a p-value that is an analysis value of the multiple logistic regression analysis is greater than a cut-off value.
